(19) United States
(12) Patent Application Publication
(10) Pub. No.: US 2009/0293211 A1
(43) Pub. Date: Dec. 3, 2009

(54) Odor Detecting Toothbrush

(76) Inventor: Marc Spungin, Palm Beach Gardens, FL (US)

Correspondence Address: MCHALE & SLAVIN, P.A., 2855 PGA BLVD, PALM BEACH GARDENS, FL, 33410, US

(21) Appl. No.: 12/129,242

(22) Filed: May 29, 2008

(51) Int. Cl. A46B 9/04; A46B 15/00; A46B 5/02; A61B 5/08

(52) U.S. Cl. 15/3; 15/105; 15/143.1; 15/167.1; 600/530

(57) ABSTRACT

The present invention is directed to a dental cleaning device having, in combination, a toothbrush and a gas detecting system. The tooth brush has a brush portion and a handle portion. The brush portion of the toothbrush has dental cleaning devices allowing the user to clean teeth. The odor detection system is integrated into the toothbrush and generally comprises a gas sensor indicator for indicating presence of predetermined gas. The system allows the user to brush their teeth and either simultaneously or at differing times, detect any odors associated with bad breath. The device further allows the user a mechanism to know when bad breath causing odors are eliminated.

### FIELD OF INVENTION

The invention relates to oral hygiene, more specifically to a toothbrush in combination with an odor detecting system so as to provide a dental cleaning device that is capable of detecting odor produced from bacteria located in the oral cavity.

### BACKGROUND OF THE INVENTION

Proper oral hygiene is considered paramount to the development and maintenance of a strong and healthy mouth. In addition to professional dental care and regular cleanings, proper and effective teeth brushing removes the sticky, colorless biofilm known as plaque produced mostly by bacteria. Removal of plaque through brushing prevents formation of cavities and periodontal diseases. Failure to remove plaque on a regular basis results in the build-up and thickening of the biofilm resulting in various dental related diseases, such as gingivitis, an early form of gum disease. Left untreated, gingivitis can progress into a more advanced disease such as periodentitis, ultimately resulting in tooth loss.

Proper brushing technique performed as a daily regiment is considered an effective method of maintaining healthy teeth and gums. Moreover, proper brushing is considered one of the best strategies for removing bacteria which builds within a person's mouth and is known to contribute to halitosis. For roughly 90% of the population, bad breath can be attributed to odor associated with bacteria that naturally populates within the mouth. Typically, bacteria produce a range of substances that are associated with bad breath, particularly hydrogen sulfide, methyl mercaptan, and dimethyl sulfide. Owing to the fact that these compounds evaporate readily they are referred to as volatile sulfur compounds, or VSCs. In addition to these sulfur compounds, some bacteria produce other odor causing substances, including cadaverine, putresince, skolate, and isovaleric acid. However, these substances are produced at extremely low levels and are unable to be detected by the human nose. As a result, the noxious smell associated with bad breath is mostly attributed to VSCs.

Although bad odor emanating from a person's mouth results in socially embarrassing situations, it is a natural part of the living processes of the human mouth. The sulfur compounds responsible for odor are the result of waste materials produced by bacteria that grow best in environments lacking oxygen. As a person ingests food in an effort to obtain essential nutrients, bacteria that live in the mouth feed as well. Proteins found in dead skin cells or in salvia can also be a source of food for bacteria. For those individuals that do not brush or floss regularly, food particles leftover in the mouth can be a further source of food for the bacteria, producing greater sources of waste production and ultimately sulfur compounds.

Not all bacteria located in a person's mouth cause bad breath. The problem of bad breath occurs when the odor carrying bacteria or their food sources are not properly removed. Removal of excess food particles through brushing would prevent bacteria from producing excess wastes. Moreover, improper brushing and flossing results in accumulation of plaque. Although plaque accumulation does not directly result in sulfur odors, accumulation provides a good oxygen depleted environment allowing odor causing bacteria to dominate over non-odor causing bacteria. Proper brushing removes the plaque buildup; flossing removes bacteria located at the gum lines. Good dental hygiene plans take advantage of both techniques in combination.

Despite regular and proper brushing, the possibility of bad breath remains. Even more difficult is determining odor levels for one's own breathe. First, determining if your own breath is foul can be difficult as a person sometimes has difficulty smelling and/or detecting odors from their own breath. Second, asking an independent third party may not be socially acceptable or desired. Asking a third party may not provide a completely objective answer as some individuals may not be completely truthful in their answer for fear of hurting another person's' feelings. Moreover, determination of levels of odor can be subjective; some individuals have very sensitive capabilities of detecting odor while other's threshold may be very high.

Therefore, what is needed is a device that objectively determines levels of predetermined odors emanating from a person's mouth while also providing the mechanism to rid the odors and underlying cause of odor production.

### DESCRIPTION OF THE PRIOR ART

Detection of various compounds in a person's mouth is not new technology. Law enforcement agents often use breath analyzers for indirect detection of the amount of alcohol associated with person's blood in order to build a case against individuals driving while intoxicated. Such sobriety tests involve blowing air from a person's mouth into a machine. Detection of alcohol levels is accomplished using one of several methods, including use of chemical reaction with alcohol that produces color changes (Breathalyzer), infrared spectroscopy (Intoxilyzer), or chemical reactions using fuel cells (Alcosensor).

In addition, the Kiss Me Meter produced by Sung-Jun International Corporation Korea, is a small, hand held breath analyzing device marketed to allow the user to take the device anywhere the user may interact with people, such as night clubs. Upon detecting the odor, a person using this machine has few options to alleviate the problem. Other than doing nothing and risking social embarrassment, the user has limited short-term options of using mints or mouth sprays. Such options generally cover-up the problem and do not offer long-term solutions. As a result, the device fails to provide a mechanism for which the user can detect odors associated with bacteria of the mouth in combination with the option of removing the problem through brushing of the teeth.

U.S. Pat. Nos. 6,858,182 discloses a cell phone equipped with the function of measuring halitosis. The inventors recognized that talking on the phone for extended lengths of time could result in a person developing dry mouth, which in turn may result in bad breath. Therefore, the invention was developed to measure levels of odor causing halitosis as a result of periods of cell phone usage and does not provide the user with the ability to instantly remove the odor.

Thus, there is a need for an improved device, such as a toothbrush and odor detection system, in combination, that detects foul smelling odors in a person's breath while simultaneously brushing their mouth which provides the mechanism to allow a user to brush their teeth until odor and odor-causing bacteria are removed.

### SUMMARY OF THE INVENTION

The present invention is directed to a dental cleaning device having, in combination, a toothbrush, or tongue scrapper, and a gas detecting system. The tooth brush has a brush portion and a handle portion. The brush portion of the toothbrush has dental cleaning devices allowing the user to clean teeth or scrap the tongue. The odor detection system is integrated into the toothbrush and generally comprises a gas sensor, microprocessor, display unit and a power source. The system allows the user to brush their teeth and either simultaneously or at differing times, detect any odors associated with bad breath. The device further allows the user a mechanism to know when bad breath causing odors are eliminated.

In accordance with the invention, the term “odor causing compounds” is defined as the sulfur-based compounds, such as hydrogen sulfide (H₂S), methyl mercaptan (CH₃SH, or dimethly sulfide (CH₃)₂S, associated with waste products produced from bacteria located in the oral cavity.

In accordance with the invention, the term “cleaning elements” is defined to include any mechanism, either manually or mechanically driven, or materials that can be used to clean teeth, such as but not limited to, bristles, brushes, rotating cleaning heads.

Accordingly, it is a primary objective of the instant invention to provide a dental cleaning device and system that detects odor causing compounds.

It is a further objective of the instant invention to provide a toothbrush in combination with a gas sensing device in order to detect sulfur-based odors produced from bacteria in the mouth.

It is a further objective of the instant invention to provide a tongue scrapper in combination with a gas sensing device in order to detect sulfur-based odors produced from bacteria in the mouth.

It is yet another objective of the instant invention to provide a toothbrush in combination with a gas sensing device which allows the user to simultaneously brush their teeth and detect odors in the mouth, providing a mechanism in which the odor caused by the odor-producing bacteria can be reliably and objectively removed.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 represents a frontal view of the toothbrush.

FIG. 2 illustrates the back view of the toothbrush.

FIG. 3 illustrates toothbrush in combination with the odor detecting system.

FIG. 4A, an embodiment of the odor detection system illustrating the inner lumen of the handle portion housing a tube in communication with the air inlet aperture and gas sensor.

FIG. 4B, cross section of the handle portion.

FIG. 5A is a block diagram of the odor detecting system, illustrating multiple gas sensing detectors.

FIG. 5B a block diagram of the odor detecting system, illustrating a single gas sensing detector.

FIG. 6A illustrates a three light display system.

FIG. 6B illustrates a display panel.

FIG. 6C illustrates a simple manner of displaying detection of a predetermined gas, using words to alert the user of negative levels of detection of an odor.

FIG. 6D illustrates a simple detection display, using words to alert the user of positive presence and detection of predetermined odors.

FIG. 6E illustrates symbolic communication of odor detection, using a smiley face to indicate no amounts of odor detected.

FIG. 6F illustrates symbolic communication of odor detection, using a face showing a frown to communicate detection of levels of a predetermined gas.

FIG. 6G illustrates use of horizontal bars to graphically represent various levels of detection of a predetermined odor from a user's breath.

FIG. 6H illustrates use of vertical bars to graphically represent varying levels of detection of a predetermined odor from a user's breath.

### DETAILED DESCRIPTION OF THE INVENTION

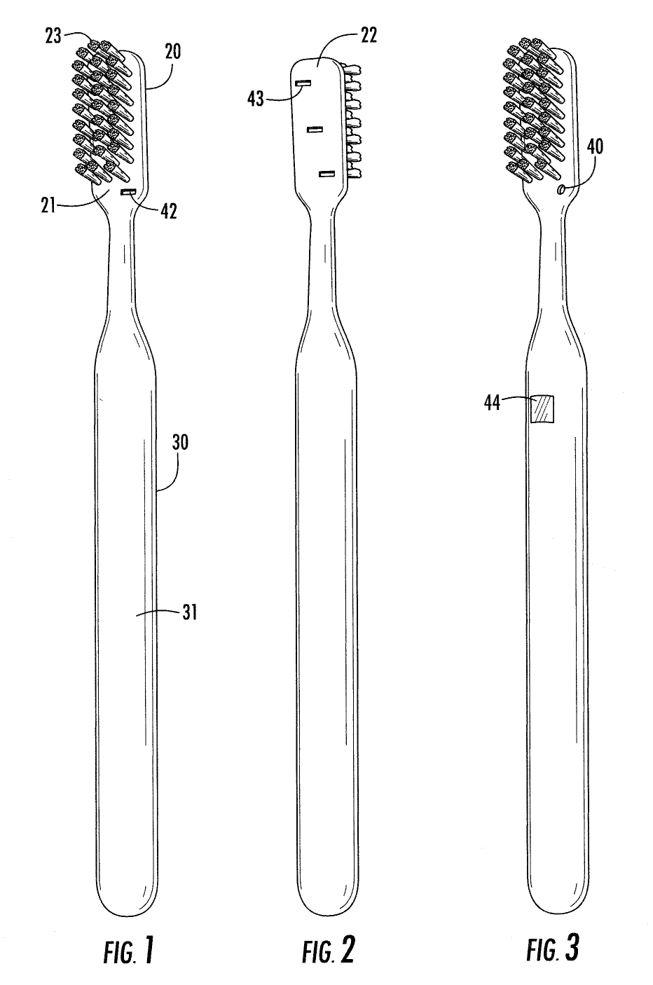

FIG. 1 illustrates a front view of toothbrush 10. As illustrated in the figure, toothbrush 10 has a brush portion 20. The brush portion 20 has a front surface 21 and a back surface 22 (See FIG. 2). Extending outwardly with respect to the front surface is a plurality of bristles 23. The back surface 22 is substantially flat. Attached to brush portion 20 an elongated handle portion 30. The elongated handle portion 30 has an outer surface 31 which defines a grip. Housed inside the elongated handle is an odor detecting system. As illustrated in FIG. 3, the odor detecting system includes at least one gas sensor 40 and at least one indicator 44 for indicating presence of predetermined gas. In another embodiment, in place of a plurality of bristles, the brush portion could include a substantially grooved surface which acts as a tongue scrapper.

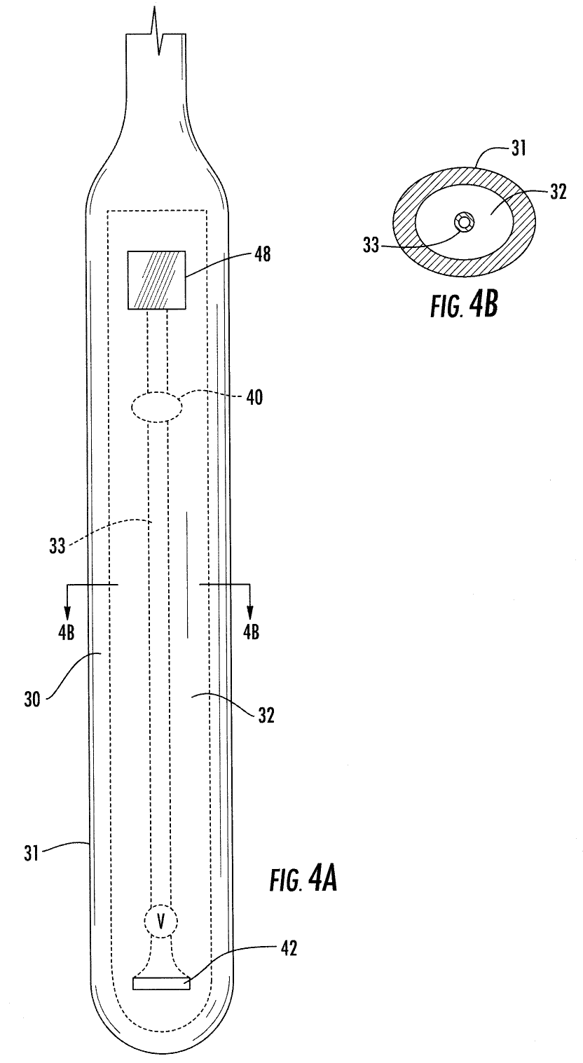

FIG. 4A illustrates a particular embodiment of the odor detecting toothbrush. The lumen 32 of the elongated handle portion 30 housing the odor detection system has an inner tube 33. Inner tube 33 connects the air inlet apertures located on the handle with the gas sensors positioned at a particular location within the toothbrush. The inner tube further has a valve, providing air flow in single direction. In this configuration, the user may exhale into the apertures positioned along the handle portion and at a distance from the brush portion. This embodiment further provides a mechanism to determine levels of odor causing compounds independently of brushing one's teeth or using the brush portion.

As an individual uses the toothbrush their breath is exhaled from the mouth into one or more air intake apertures 42. Additionally, the user can directly exhale into the air inlet apertures. In addition to exhaling into the air inlet openings, the toothbrush may contain a device, such as but not limited to a fan or pump, such that when a user engages, air exhaled into the toothbrush is moved into the brush so as to flow over sensors located at a distance from the inlet apertures. Odor casing compounds may be detected by use of an odor detection system integrated within the toothbrush. FIG. 5A illustrates an embodiment of the odor detecting system that is integrated into the toothbrush. The system comprises the parts of a sensor 40, microprocessor 46, display unit 48 and power supply 19. Air inlet apertures 42 provide the air flow necessary for detection. As illustrated, multiple inlet apertures coupled to multiple gas detectors are used. FIG. 5B illustrates another preferred embodiment in which the odor detection system contains a single gas sensor without a power supply. Although both embodiments illustrate several air inlet apertures, it can be appreciated that a single air inlet may be used.

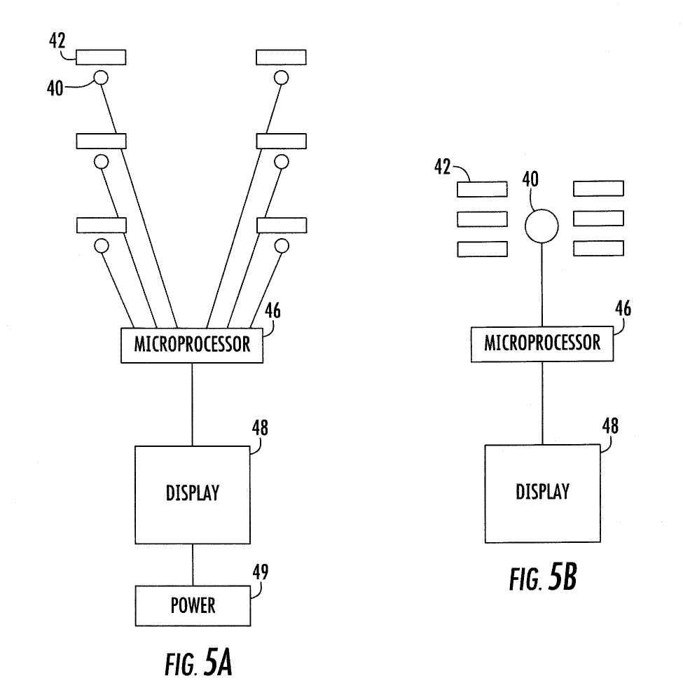

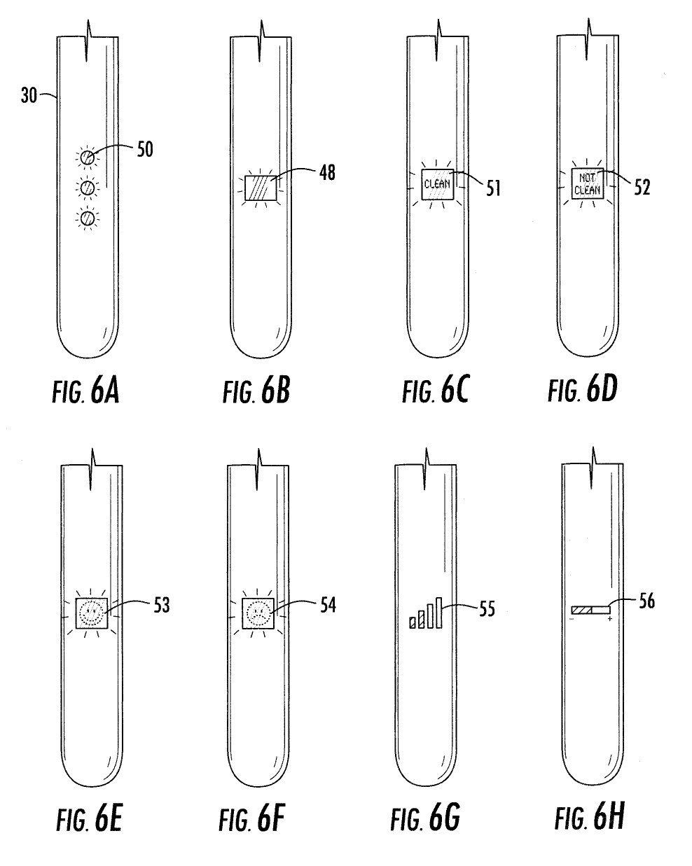

Upon brushing one's teeth, odors caused by odor producing bacteria enter the toothbrush through the one or more intake apertures. As illustrated in FIG. 1, the intake apertures are located in the brush portion. Such arrangement allows the user to simultaneously brush their teeth and detect the level of odor in their breath. In addition, the air intake apertures could be located along the handle as well, FIG. 4A, allowing the user to directly blow into the apertures in order to receive a reading. As the user's breath enters the air intake apertures, one or more gas sensors detect the presence of sulfur or other predetermined compounds. Any type of sensor that detects the presence of sulfur associated with VSCs could be used. For example, Kiss Me Meter manufactured by Sun-Jin International Corporation uses a microelectrochemical system (MEMS) gas sensor as the detecting method in that device. In addition to this, a semiconductor gas sensor, as described by U.S. Pat. No. 6,858,182, may also be used, the contents of which are incorporated herein by reference. Connected to the gas sensors is a microprocessor 46. The microprocessor may be powered by any means, such as by a low power consumption source such as a lithium battery. Microprocessor 46 processes all the data from the gas sensor; the results are measured and displayed on the indicator device 44 or display unit 48. Indicator device can be one or more lights that are programmed to display light in the presence of a predetermined odor, FIG. 6A. Additionally, a display unit 48 connected to the microprocessor could be used as well, FIG. 6B. In a preferred embodiment, the display unit could be a LCD display unit.

Levels of odor can be displayed in various manners, including graphically and/or using alphanumeric representations. For example, the display unit could display simple “clean” to depict low levels of odor and “not clean” to depict high levels of odor production, FIGS. 6C and 6D respectively. In addition to words, symbols, such as a happy face designating the presence of and a face depicting a frown to designate the presence of odor, FIGS. 6E and 6F. The display unit could also display information received from the microprocessor in varying degrees. For example, a multiple bar level system FIG. 6G, such as those used on cell phones to illustrate phone signal, may be used to help the user determine varying levels of odor strength. Additionally, a bar/rod level indicator FIG. 6H, also commonly used in cell phone technology to communicate phone signal or battery levels, may be used. Although FIG. 6 depicts several methods of communicating odor levels, any manner that clearly informs the user that their breath that has been analyzed and communicates the results of the analysis could be used. In addition, the presence of odor can be indicated by use of sound emitting devices, such as piezo audio indicators, piezoelectric crystals, piezoelectric sound components, or the like.

With a display system located in the toothbrush, the user has a mechanism that allows them to receive an objective determination of the levels of odor in their breath with out having to resort to self-detection methods or embarrassingly asking a third party. Detection could be performed simultaneous to brushing of the teeth, allowing the user to continually brush their teeth until the display unit displays a positive, or lack of odor, signal.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

I claim:

1. A dental cleaning device comprising, in combination, a brush portion, said brush portion having a front surface and a back surface, said brush portion having a plurality of bristles extending outwardly with respect to said front surface, said back surface having a substantially flat surface, an elongated handle portion, said elongated handle portion having an outer surface, said outer surface defining a grip, said elongated handle portion including an odor detecting system, said odor detecting system including at least one gas sensor and at least one indicator in electrical communication with said at least one gas sensor for indicating presence of a predetermined gas.

2. The dental device according to claim 1 wherein said handle portion further comprises an inner lumen, said inner lumen housing said odor detecting system.

3. The dental cleaning device according to claim 2 further includes inlet and/or outlet apertures.

4. The dental device according to claim 3 wherein said inlet aperture and said outlet aperture are positioned in the head region.

5. The dental device according to claim 3 wherein said inlet aperture and said outlet apertures are positioned along said handle region.

6. The dental device according to claim 3 further comprises a pump, for causing air flow into said inlet aperture across said at least one air sensor and out of said outlet aperture.

7. The dental device according to claim 1 wherein said at least one indicator includes one or more lights displayed on said handle portion.

8. The dental device according to claim 1 wherein said indicator includes a sound emitting device located on said handle portion.

9. The dental device according to claim 1 wherein said odor detecting system further comprises a microprocessor said microcompressor in electrical communication with said at least one gas sensor and said at least one indicator.

10. The dental device according to claim 1 wherein said back surface of said brush portion is substantially grooved so as to function as a tongue scrapper.

11. A toothbrush equipped with a system for measuring odor causing components comprising, in combination, a brush portion, said brush portion having a front surface and a back surface, said front surface of said brush portion further having a plurality of bristles extending outwardly with respect to said front surface, said back surface having a substantially flat surface, a handle portion connected to said brush portion and having an outer surface defining a grip and an inner lumen, said handle portion housing at least one or more gas sensors positioned to receive one or more breaths from a user's mouth through air inlet apertures, said user's breath moving over said sensors and exiting the toothbrush through outlet apertures, a microprocessor, said microprocessor being electrically connected to said one or more gas sensors and programmed to indicate levels of odor causing compounds to a display unit, said display unit electrically connected to said microprocessor and capable of displaying information received from said microprocessor.

12. The toothbrush equipped with a system for measuring odor causing components according to claim 11 wherein said air inlet and/or outlet apertures are positioned within said handle portion.

13. The toothbrush equipped with a system for measuring odor causing components according to claim 11 wherein said display unit exhibits values of odor causing components using one or more lights.

14. The toothbrush equipped with a system for measuring odor causing components according to claim 11 wherein said display unit includes LEDs for exhibiting the presence of odor causing components.

15. The toothbrush equipped with a system for measuring odor causing components according to claim 14 whereby said display unit exhibits values of odor causing components using alphanumeric characters.

16. The toothbrush equipped with a system for measuring odor causing components according to claim 14 whereby said display unit exhibits values of odor causing components symbolically.

17. The toothbrush equipped with a system for measuring odor causing components according to claim 11 further comprises a power supply unit.

18. The toothbrush equipped with a system for measuring odor causing components according to claim 17 wherein said power supply unit includes at least one battery.

19. The toothbrush equipped with a system for measuring odor causing components according to claim 11 further comprising an inner tube located within said lumen of said handle portion, said inner tube in communication with said air inlet apertures and said gas sensors.

20. A dental device equipped with a system for measuring odor causing components comprising, in combination, a brush portion, said brush portion having a substantially grooved surface so as to function as a tongue scrapper, a handle portion connected to said brush portion and having an outer surface defining a grip and an inner lumen, said handle portion housing at least one or more gas sensors positioned to receive one or more breaths from a user's mouth through air inlet apertures, said user's breath moving over said sensors and exiting the toothbrush through outlet apertures, a microprocessor, said microprocessor being electrically connected to said one or more gas sensors and programmed to indicate levels of odor causing compounds to a display unit, said display unit electrically connected to said microprocessor and capable of displaying information received from said microprocessor.
